(19) United States
(12) Patent Application Publication
(10) Pub. No.: US 2007/0276246 A1
(43) Pub. Date: Nov. 29, 2007

(54) Ultrasound image acquisition guiding device and method

(76) Inventor: Shengtz Lin, Cupertino, CA (US)

Correspondence Address: Bo-In Lin, 13445 Mandoli Drive, Los Altos Hills, CA, 94022, US

(21) Appl. No.: 11/254,074

(22) Filed: Oct. 18, 2005

Related U.S. Application Data
(60) Provisional application No. 60/664,461, filed on Mar. 22, 2005.

(51) Int. Cl. A61B 8/00

(52) U.S. Cl. 600/444; 600/437; 600/443

(57) ABSTRACT

In an embodiment, there is an apparatus for facilitating ultrasound medical image acquisition. There is a guide to help guide an ultrasound probe during a scanning operation. The guide resists at least some undesired movement of the ultrasound probe. The at least some undesired movement is otherwise possible, without the guide, under freehand probing. In another embodiment, there is a method for facilitating ultrasound image acquisition. The method includes providing a guide; guiding an ultrasound probe during a scanning operation using the guide, including resisting at least some undesired movement of the ultrasound probe; wherein the at least some undesired movement is otherwise possible, without the guide, under freehand probing. The guide and guiding are especially beneficial for three-dimensional or panoramic ultrasound imaging.

### RELATED APPLICATION(S)

The present patent application is related to and claims the benefit of priority from commonly-owned U.S. Provisional Patent Application No. 60/664,461, filed on Mar. 22, 2005, entitled “Ultrasound Image Acquisition Guiding Device and Method”, which is hereby incorporated by reference in its entirety for all purposes.

### BACKGROUND

The present invention relates to medical imaging. Embodiments of the present invention are especially suitable for ultrasound imaging, especially 3-Dimensional (3D) ultrasound imaging or panoramic imaging.

Medical imaging systems are known. For example, ultrasound imaging is discussed in U.S. Pat. Nos. 6,080,108, 6,108,439, 6,248,071, 6,338,716, 6,780,153, 6,783,497, 6,824,514, 6,117,080, 6,540,681. These and any suitable other existing references that mention medical imaging systems, e.g., ultrasound imaging systems, can be referred to for conventional details that need not be described in the present document.

For further examples, conventional commercially available ultrasound systems include the enVisor ultrasound system available from Koninklijke Philips Electronics N.V. (see, e.g., “EnVisor Series Ultrasound System User Guide, M2540-30000-ug-03 Rev B.0” from the manufacturer), the “LOGIQ Book” ultrasound system available from GE Medical Systems (see, e.g., “LOGIQ Book Basic User Manual, R2.0.0, Direction 2321062-100, Rev. 3” from the manufacturer), and systems that use a familiar pivoting (sector-scanning) style of probe, e.g., probes such as the RAB 4-8 Probe for the Voluson 730 system, available from GE Medical Systems.

Conventional ultrasound systems, e.g., 3D ultrasound systems generate a 3D image by combining 2-dimensional (2D) images (“slices”) that are successively taken by a handheld probe that is moved or rotated across body tissue. There are difficulties and/or high costs associated with obtaining consistently good results with many such systems. For example, the 3D acquisition systems and methods of the GE ‘LogiqBook’ and Phillips ‘enVisor’ systems require for good image acquisition that a human operator manually move the probe across body tissue at a smooth and consistent rate in a freehand fashion. The movement is intended to be in a z direction at a constant rate such that successive images, each extending in the x and y direction, can be combined to form a volume. Such a freehand task is not easy and requires much training and practice just to achieve a certain level of tolerable adequacy. Such freehand scanning is also called “easy 3D”. With the freehand “easy 3D” style of image acquisition, a typical user cannot consistently control or maintain a consistent acquisition scan distance, scan speed, scan direction, probe scan angle etc. Such lack of consistency all too frequently makes the 3D reconstruction result poor or altogether unacceptable. Not only is the required operator training very significant and extensive, but the acquisition process and result also varies undesirably much from patient to patient and across different parts of the body.

### SUMMARY OF THE INVENTION

What is needed are apparatuses and methods to improve ease and reliability of 3D ultrasound image acquisition.

According to some embodiments of the present invention, there is an apparatus or a method that improves ease of obtaining consistent image acquisition with ultrasound systems, e.g., including one or more models of existing 3D ultrasound systems. For example, such an apparatus can be a low-cost apparatus that interfaces with an existing ultrasound system's freehand “easy 3D” style of probe to facilitate smooth and constrained moving of the probe. For example, such an apparatus can be easy to use such that extensive training of its user is not required.

According to some other embodiments of the present invention, there is an ultrasound system and method that is capable of obtaining consistent image acquisition. Such a system and method include a conventional ultrasound system and method that are configured and embodied to include the apparatus or method described in the previous paragraph.

According to some embodiments of the present invention, there is an apparatus for facilitating ultrasound medical image acquisition. The apparatus includes at least one member configured to help guide an ultrasound probe during a scanning operation, to resist at least some undesired movement of the ultrasound probe. The at least some undesired movement is otherwise possible, without the member, under freehand probing. Optionally, the apparatus may include a motor drive configured to move the ultrasound probe relative to skin during a scanning operation. Optionally, the apparatus does not include such a motor drive. Optionally, the apparatus may include at least one sensor, the at least one sensor being configured to detect at least a first and a second position of the ultrasound probe during a scanning operation, and to communicate detection of the first and second position of the ultrasound probe to an ultrasound information processing system.

According to some embodiments of the present invention, there is a method for facilitating ultrasound image acquisition. The method includes providing a guide; guiding an ultrasound probe during a scanning operation using the guide, including resisting at least some undesired movement of the ultrasound probe; wherein the at least some undesired movement is otherwise possible, without the guide, under freehand probing.

### BRIEF DESCRIPTION OF THE DRAWINGS

In order to more extensively describe some embodiment(s) of the present invention, reference is made to the accompanying drawings. These drawings are not to be considered limitations in the scope of the invention, but are merely illustrative.

FIG. 1 is a schematic diagram of an ultrasound system according to a particular embodiment of the present invention

FIG. 2 is a schematic diagram that shows a probe bracket that is configured to be connected on-demand to an ultrasound probe.

FIGS. 3A and 3B are schematic diagrams that respectively show a top view and a side view of an embodiment of the guide device of FIG. 1.

### DETAILED DESCRIPTION OF SPECIFIC EMBODIMENTS

The description and the drawings of the present document describe examples of some embodiment(s) of the present invention and also describe some exemplary optional feature(s) and/or alternative embodiment(s). It will be understood that the embodiments described are for the purpose of illustration and are not intended to limit the invention specifically to those embodiments. Rather, the invention is intended to cover all that is included within the spirit and scope of the invention, including alternatives, variations, modifications, equivalents, and the like. Similarly, use of language in the present document is not intended to be misinterpreted to limit the scope of the invention.

According to some embodiments of the present invention, there is an ultrasound system that includes a probe. For example, the ultrasound system may be any conventional ultrasound system. For example, the ultrasound system may be a 3D ultrasound system. For example, the probe may be a freehand “easy 3D” probe that is meant to be moved in translational motion in the z direction across body tissue. The probe is attached to a probe guide. The probe guide is by itself also an embodiment of the present invention. The probe guide is configured to resist at least some types of movement (e.g., prevent gross movement beyond a predetermined amount and type) by the probe other than according to one or more predetermined permitted movement(s).

For example, if the permitted movement is a purely translational motion in a z direction, then the guide may be configured (e.g., by fixed designer choice; e.g., by elective user adjustment) to prevent gross motion in one or more or all modes of motion other than translational motion in a z direction. Further, in the example, the guide may restrict or delimit the extent of such permitted translational motion in the z direction. Improved ease of traversal by the probe is obtained. The attaching of the probe to the probe guide is preferably on an on-demand basis by the operator of the system, such that a probe may easily be used either freehand or, alternatively, with the probe guide.

In some embodiments for which the permitted movement is purely translational movement in the z direction, the probe guide is configured to resist movement by the probe in some or all modes of spatial motion other than the permitted translation in the z direction. (For example, the total modes of motion may be the following six modes of motion: translation in x, y, and z; and rotation in pitch, yaw, and roll.) In the some embodiments, the probe guide is configured to resist movement in at least two, or at least three, or at least four, or all of the five prohibited modes of motion (i.e., the five modes other than translation in the z direction).

In other embodiments, the permitted movement follows a program (e.g., a curved permissible path) that includes multiple modes of motion in combination. The probe guide (e.g., a curved track) can be said to resist movement by the probe (e.g., prevent gross movement) other than according to a predetermined permitted movement.

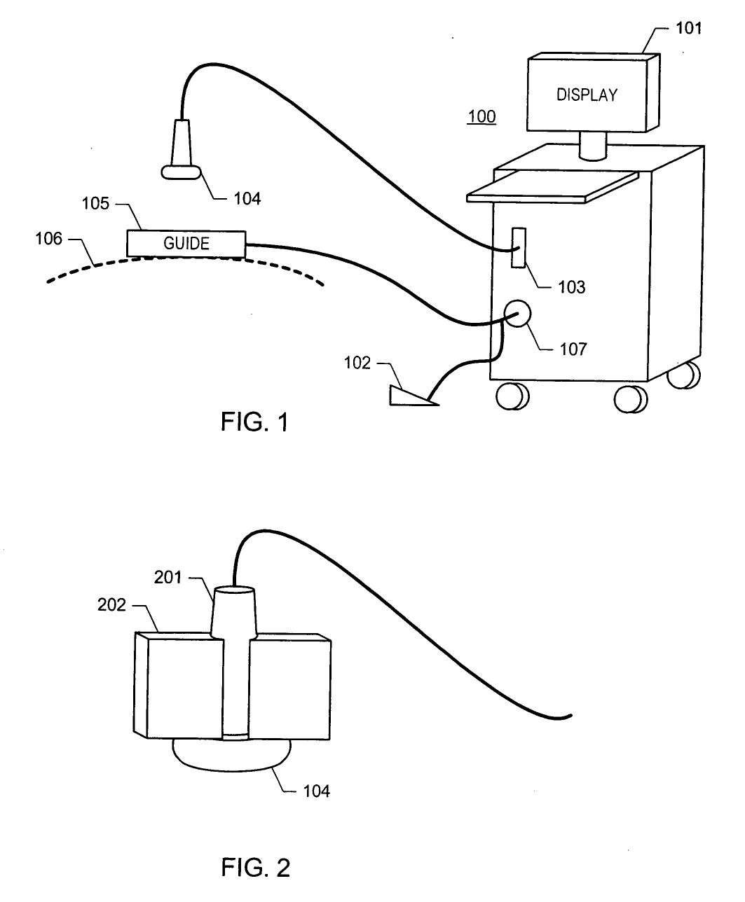

FIG. 1 is a schematic diagram of an ultrasound system according to a particular embodiment of the present invention. An ultrasound system 100 includes a display 101, a foot switch input port 107, a foot switch 102, a probe input port 103 and a probe 104. For example, the ultrasound system 100 may be a conventional ultrasound system and include any combination of components that is found on conventional ultrasound systems (so long as the components do not necessarily prevent use of a guide device, according to embodiments of the present invention, that is meant to be used with the ultrasound system 100). The system further includes a guide device 105, according to the present invention. The guide device 105 is itself another embodiment of the present invention. The guide device 105 is attached to the probe 104. The attaching is preferably a user-removable attaching, such that the probe 104 may either be used freehand, or, alternatively, with the guide device 105. The guide device 105 is preferably used especially for 3D scanning or panoramic imaging of a patient 106.

The guide device 105, in general, is configured to attach to the probe 104 using any competent mechanism whatsoever. For example, any type or combination of temporary or permanent or semi-permanent clamp and/or fastener and/or strap and/or adhesive and/or geometric feature and/or friction mechanism and/or the like and/or any other type of competent coupler or couplers whatsoever may be used. According to one embodiment, the guide device 105 is configured to include a connector that can connect to an intermediate connector (e.g., to an adaptor). The intermediate connector is configured to, in turn, connect to one or more possible sizes or models of probes 104. For example, the intermediate connector may be a clamp or fastener that can accommodate either a specific size and shape of probe handles, or a range of different sizes or shapes of probe handles. Optionally, any of multiple different intermediate connectors may be chosen that is most appropriate for a particular (range of) size or shape or design of a particular model of probe 104. The preferred implementation of the guide device 105 can be referred to as a “universal” guide device 105; it can be used with a variety of model probes 104. The universal guide device 105 can be coupled to any of one or more sizes or types or models of probe 104, including models of probe 104 that were designed originally only for freehand operation and designed originally without any consideration of the guide device 105. Of course, if desired, a guide device 105 can also be configured specifically for a particular model probe 104, for example, by integrating the functionality of the intermediate connector into the probe 104 itself (i.e., by configuring the probe 104 and/or the guide device 105 so that the probe 104 itself is connectible or engageable to the guide device 105 without an intermediate connector).

For example, FIG. 2 is a schematic diagram that shows a probe bracket 202 (e.g., a type of intermediate connector) that is configured to be connected on-demand, e.g., via a releasable (e.g., quick release) clamp (not shown), to the probe 104, e.g., at a probe handle 201 of the probe 104.

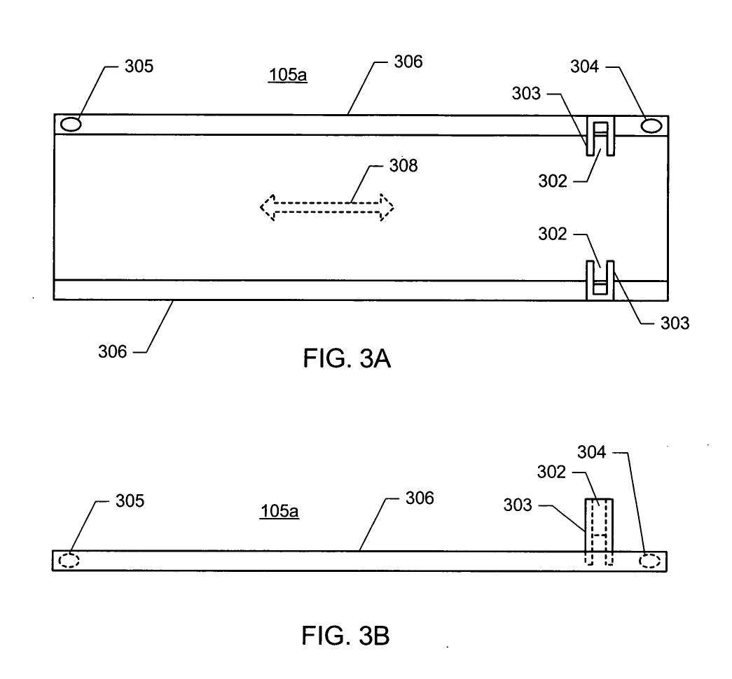

FIGS. 3A and 3B are schematic diagrams that respectively show a top view and a side view of an embodiment 105a of the guide device 105 of FIG. 1. The guide device 105a includes a guide, e.g., one or more rail(s) 306 that at least help define a permitted movement for a probe. For example the rail(s) 306 may be any of different possible types. For example, the rails 306 may define substantially merely a linear motion in a z direction, as schematically shown by an arrow 308. Alternatively, the rails 306 may define a curved path (not shown) of motion; the curved path may reflect a desired program of permitted translational motion and/or rotational motion. For example, the rail(s) 306 may be curved in any manner, e.g., with any curvature to fit skin contour. For example, the curvature may include a constant or varying-with-position radius of curvature in any of one or more dimensions. In FIG. 3, two rails 306 are depicted, as an example.

For example, the guide device 105a may include a slider 303 that slides along the rails 306. The probe bracket 202 is attached to the probe 104 to couple the probe 104 to the guide device 105a. For example, in the shown embodiment, the probe bracket connects to the slider 303, e.g., via a connector, e.g., via a releasable (e.g., quick-release) connector, e.g., via a simple guide slot 302 defined by a connector of the slider 303). For example, the guide slot 302 may simply be an unsecured slot that accepts and seats the probe bracket using only gravity (and operator hand pressure) and the geometric fit between the probe bracket and the slot—e.g., without using any clamp or click-in. Alternatively, a clamp or click-in tab may be included. The probe 104 (attached to the bracket 202 and situated in the slider 303) can be manually or automatically (motor driven) moved along the rail 306 at a speed that is suitable for the application and for the ultrasound system in question.

For a linear guide designed for purely translational motion, the guide is preferably configured to resist motion (e.g., prevent gross motion) in at least one or two or three or four or all five modes of motion other than the permitted mode of motion. Or, if the permitted motion follows a program (e.g., a curved track) that includes multiple modes of motion in combination, then the guide can be said to resist motion (e.g., prevent gross motion) other than according to a predetermined permitted movement.

Optionally, there are two micro switches 304 and 305 at each side of the slide rail 306 to detect the start and stop of the movement. These two switches connect to the system via, e.g., the system's input port 107 for a foot switch as an example, or via any other input of the system. (As shown in the example of FIG. 1, optional Y-shaped wiring (e.g., a Y-adaptor) may be included or used, in place of default straight wiring, to enable the foot switch 102 to remain simultaneously connected to the ultrasound system or to remain simultaneously operative.) When the system is in 3D acquisition mode, at the instant when user moves the slider, one switch will trigger and the system will then start to acquire the image in the cine buffer, and the system will stop when the other switch detects the end of the slider traveling. In the cine image buffer, the image frames are acquired across a distance defined by the separation of the two switches, which may be defined by the guide rails' length. Optionally, the separation between the two switches may be user-adjustable manually or via software. Optionally, there are an array of switches, and the designation of which switches to use as the starting and ending points is similarly adjustable, e.g., manually via manual switches on the guide device 105a or via software. The probe bracket is configured to allow substantially only permitted freedom of movement against the rails 306. For z-direction translation as the permitted freedom of movement, the acquired images will have a substantially constant linear movement and will not have substantial rocking angle change. Optionally, the guide device 105a may permit a certain amount of up-and-down (e.g., y-direction) movement of the probe, to help the operator provide good contact with the patient's skin surface by following skin contour. The user may slide the probe faster or slower, and as long as it is almost in constant speed, the system won't cause intolerable 3D image distortion. Or, the probe guide 105a may be configured to include a suitably precise (according to design goal) motor drive to ensure the desired degree of speed constancy. Any competent type of motor drive whatsoever may be used, whether it uses electrical power (preferred) and/or mechanical-stored-energy, pneumatic power, and/or the like and/or any other type of mechanism or principle.

The slider may be configured to provide some resistance (but not prevention) to the permitted manual linear movement. Then, it will be easier, as a matter of ergonomics, for the human operator to obtain a more constant sliding speed. The resistance may be provided by any competent mechanism. The resistance may be operator adjustable, e.g., manually or, optionally, via software. The resistance may be provided, e.g., via simple sliding friction and/or via a gear (or wheel) that engages gear teeth (or wheel track) on the rail and that drives a small frictional or other load and/or via any other competent mechanism.

With this 3D guide sitting on top of patient skin, and acquiring the image with the above description, a multiple-slices ultrasound image along the rail dimension (e.g., z-dimension) will be stored in the system's cine memory. Since each slice is already in 2D format, totally the 3D volume of ultrasound information can be processed, e.g., using conventional algorithms or ultrasound systems. The system can use surface rendering to construct, e.g., a baby face, gallbladder disease or breast cyst or the like; or volume rendering for the 3D tumor extraction or the like.

The patient contact surface of the probe guide 105a can be made as a curve shape in geometry to fit better with the skin contour, such as for baby face image acquisition.

The device can also implement an optional sensor, e.g., a linear optical encoder, to report the step of movement for the slider to the system. For example, there may be optional sensors (e.g., tickmarks on a rail 306, to be read by a sensor on the slider 303) configured to detect positions at least every 1 millimeter apart on the guide.

To facilitate good contact between the probe and the patient, the device can have a tiny spring(s) at the bottom of the probe insertion slot 302 so the user can apply a slight force to make a better skin contact. (The patient contact generally will utilize acoustic Gel for coupling.) Additionally, or alternatively, the device can have a small spring(s) that provides a slight push of the probe into the patient; for example, the small spring(s) can engages the probe bracket 202 after the probe bracket 202 optionally “clicks in” to the slider 303; the “click in” may be via a click-in tab on either the probe bracket 202 or the slider 303.

Any 3D surface and volume rendering algorithm may be used in the system software, e.g., any conventional algorithm or the like or any other competent algorithm.

Embodiments of the present invention obtain easy 3D image acquisition for any conventional ultrasound system, including ones implemented without any thought or knowledge of the probe guide device according to embodiments of the present invention. For example, a probe guide can be configured to realize any probe trajectory that is already compatible with the expectations or requirements of an existing ultrasound system and to provide information of a type and in a manner that is already expected by an existing ultrasound system.

Embodiments of the present invention can also include, or be configured to work with, ultrasound systems specifically designed with awareness of the embodiments of the present invention. For example, a probe guide can be designed to realize a new desired trajectory or other device parameters or to capture various information, and an ultrasound system can be specifically programmed to be able to handle that probe trajectory or other device parameters and those captured information. For example, a probe guide can present information to the ultrasound system to indicate the trajectory of the probe guide and/or other device parameters and/or to indicate captured information. Indication of the trajectory and other device parameters may be an identifier(s) indicating the correct member(s) of a predetermined list of trajectory or device parameters, with the list being already available to, or within, the ultrasound system. Or, indication of the trajectory and other device parameters may be via a specification of a new trajectory and/or new parameters, and/or new combination of trajectory and/or parameters, to the ultrasound system, e.g., according to any competent (e.g., predetermined) format for specifying such parameters. Captured information can include probe position information for specific images, general position or speed information, or the like.

In 3D image acquisition, the memory buffer requirement can easily be high. For example, the requirement may be 256 MB or higher, especially when the user moves the probe slowly. Optionally, the probe guide 105a can include position sensors, e.g., an array of switches along the rails, e.g., spaced every 1 cm apart. When the system detects the 1 cm pulse (perhaps through the foot switch input port), it can immediately calculate the probe moving speed (in terms of frame per mm) and perform real time preprocessing to reduce the total number of frame images to be retained over a certain predefined distance. For example, if a 3D algorithm being used requires one frame per half millimeter, then the preprocessing can ensure that no more than one frame per half millimeter are retained, or no more than some fixed multiple of that amount of frames is retained after the real time preprocessing. The reduction may be by averaging nearby frames or by dropping unneeded frames.

The guide device 105a is intended to be in substantially fixed position relative to the patient being scanned. Any competent mechanism can be utilized to facilitate such positioning. For example, the side of the guide device 105a that contacts the patient can be of a material and/or texture having high friction (e.g., a material of, or having characteristics of, rubber). For another example, a tacky or adhesive material may be used between the guide device 105a and the patient to resist relative movement of the guide device 105a. For another example, a belt or vest or other article of clothing or the like may be donned by the patient that is configured with a hole for the probe 104 and with engagement points for permanently or (preferred) temporarily engaging the guide 105a to prevent relative movement of the guide device 105a; for example, the patient can, using the belt or vest or other article of clothing, “wear” the guide device 105a. For another example, the guide device 105a may include or be attached to a mechanical member that is secured to maintain position of the guide device 105a; for example, the member may be secured, e.g., clamped or strapped or clicked-in, to the patient's bed (e.g., via a bed's bedside railing); the mechanical member may be a swing arm, or the like, or be of any other competent design and configuration.

Specific embodiments of the present invention are further discussed below.

One example embodiment EX-1 of the present invention includes a 3D ultrasound image acquisition guiding device that accepts a probe at a slider. The slider is configured to engage a flat or curved rail. The slider can be moved along the rail while acquiring 3D image.

One example embodiment EX-2 of the present invention is like EX-1, and the movement of the slider is manual.

One example embodiment EX-3 of the present invention is like EX-1, and the movement of the slider is automatic, via a motor drive.

One example embodiment EX-4 of the present invention is like EX-1, and includes a switch that detects the start and stop of the slider and conveys the detection to the ultrasound system. The switch optionally is connected to the ultrasound system via an existing foot switch input in an existing ultrasound system, and the conveying of the detection is via footswitch signal.

One example embodiment EX-5 of the present invention is like EX-1, and the patient contact surface of the plate can be flat or curved.

One example embodiment EX-6 of the present invention is like EX-1, and the slider can have spring or cushion to provide better patient contact when pressing the probe during the scan.

One example embodiment EX-7 of the present invention is like EX-1, and the rail can have a friction slide, wheel, gear, or any combination thereof to provide resistance to help maintain the evenness movement for manual sliding.

One example embodiment EX-8 of the present invention is like EX-1, and the rail can install a motor to drive the slider automatically at a constant speed.

One example embodiment EX-9 of the present invention is like EX-1, and the rail can have a bar scale and detector such as optical encoder to provide the slider position feedback.

One example embodiment EX-10 of the present invention is like EX-1, and an optional probe bracket or clamp shell exists to couple the slider to different probe handles for better fitting.

One example embodiment EX-11 of the present invention is like EX-1, and the system software can calculate the acquired 3D image slice increment by dividing the slider traveling distance by the total number of slice over the acquisition.

One example embodiment EX-12 of the present invention is a method for acquiring acoustic medical imaging information. A guide is provided that restricts motion of a sonogram probe other than according to a range of predetermined permissible movement. The sonogram probe is moved over a trajectory, the trajectory at least in part defined by the guide. Over the trajectory, the sonogram probe obtains multiple medical images of a patient.

In some example embodiments that permit translation linear motion, the tolerances may be selected such that prohibited translation movement in one (e.g., x) or both of the x and y directions is restrained to be no more than about 0.1 mm or less than 0.2 mm or less than 0.5 mm or less than 1 mm or less than 2 mm within any predetermined particular probe traversal interval or subinterval. The subinterval or interval may be, e.g., 1 cm, or 2 cm, or 5 cm.

Another application for some embodiments of the present invention relates to panoramic image acquisition. Panoramic image acquisition is described, for example, in U.S. Pat. No. 6,540,681. Panoramic image acquisition, in two dimensions (2D), is similar to taking multiple pictures with a camera and then stitching the pictures together to have a wide-view picture. Consider a probe that takes a 2D image. The width of the probe determines the width of the 2D image. In 3D imaging, as depicted in FIGS. 1 and 2, successive 2D images (“slices”) are essentially “stacked” next to each other to synthesize a 3D volume whose width is determined by the width of the probe. In (2D) panoramic imaging, a wide view 2D image is synthesized from multiple 2D images of the same plane, to obtain an image that is wider than the probe width.

Probe guide devices and methods of some embodiments of the present invention can be adapted to facilitate accurate and consistent panoramic imaging. The probe guide of these embodiments can be as described above for 3D imaging, except that the probe is inserted into the probe guide with a turn of 90 degrees, relative to the arrangement described above for 3D imaging. For example, the image plane of the probe, at various points along the guide, remains substantially in the same overall plane. When acquiring such same-plane images with a guide such as depicted in FIGS. 1 and 3, the system can obtain multiple (2D) images e.g., substantially in a same plane, with high degree of overlapping, and software can process the images according to any competent algorithm to make an extended view ultrasound image, e.g., for breast, liver, or muscle diagnostic purposes or the like or any other purpose. Optionally, position sensors in the guide can provide position information for some or all the multiple (2D) images, to assist the software in stitching the images together (e.g., by constraining the possible region of image-stitching boundaries). Optionally, probe guide devices can be customized for the panoramic imaging task. For example, a probe guide device for panoramic imaging may be made that is narrower than similar probe guide devices for 3D imaging (e.g., from FIG. 3), if the probe to be used is narrower when turned 90 degrees (relative to its orientation when used for 3D imaging).

Again, it is to be understood that the embodiments described are for the purpose of illustration and are not intended to limit the invention specifically to those embodiments. For example, although the guide is shown in an example embodiment as a rail, any other competent mechanical configuration may be used. Any type of linkage assembly (e.g., 4-bar linkage) or arm may also or instead be used. Still other variations are within the scope of the present invention.

What is claimed is:

1. An apparatus for facilitating ultrasound medical image acquisition, the apparatus comprising:
at least one member configured to help guide an ultrasound probe during a scanning operation, to resist at least some undesired movement of the ultrasound probe;
wherein the at least some undesired movement is otherwise possible, without the member, under freehand probing.

2. An apparatus according to claim 1, wherein the at least one member comprises a flat or curved rail defining a trajectory along a surface of skin, the rail being configured to resist movement of the ultrasound probe at least in a direction along the surface that is other than the defined trajectory.

3. An apparatus according to claim 2, wherein the rail is configured to be directly engaged by an ultrasound-probe housing, the ultrasound-probe housing being capable of sliding along the rail.

4. An apparatus according to claim 2, further comprising a slider configured to slideably engage the rail, the slider being configured to couple to an ultrasound probe unit.

5. An apparatus according to claim 2, wherein the apparatus includes a resistive element that resists, but does not prevent, some motion even along the defined trajectory, the resistive element thereby improving ergonomic ability to maintain evenness of movement.

6. An apparatus according to claim 1, further comprising a motor drive configured to move the ultrasound probe relative to skin during a scanning operation.

7. An apparatus according to claim 6, wherein the motor drive is configured to move the ultrasound probe according to predetermined target speed, the predetermined target speed being used to process imaging data from the scanning operation.

8. An apparatus according to claim 1, wherein the apparatus lacks a motor drive for driving the ultrasound probe, and wherein the apparatus is suitable for the ultrasound probe to be manually moved, with movement guidance being contributed by the apparatus.

9. An apparatus according to claim 1, further comprising at least one sensor, the at least one sensor being configured to detect at least a first and a second position of the ultrasound probe during a scanning operation, and to communicate detection of the first and second position of the ultrasound probe to an ultrasound information processing system.

10. The apparatus according to claim 9, wherein the first and second positions are indicative of, respectively, a start and a finish of a desired scanning operation.

11. The apparatus according to claim 9, further comprising a communication adaptor configured to communicate the detection of the first and second position of the ultrasound probe to the ultrasound information processing system via a foot-switch input of the conventional ultrasound information processing system.

12. The apparatus according to claim 1, wherein the member is configured to help guide the ultrasound probe, with the ultrasound probe oriented for at least one of a three-dimensional scanning operation or a panoramic scanning operation.

13. An apparatus according to claim 1, further comprising:
a medical ultrasound imaging system that includes a computer system, ultrasound image acquisition and processing software; and
the ultrasound probe.

14. A method for facilitating ultrasound image acquisition, comprising:
providing a guide;
guiding an ultrasound probe during a scanning operation using the guide, including resisting at least some undesired movement of the ultrasound probe;
wherein the at least some undesired movement is otherwise possible, without the guide, under freehand probing.

15. A method according to claim 14, wherein the guide comprises a flat or curved rail defining a trajectory along a surface of skin, and the at least some undesired movement includes movement of the ultrasound probe at least in a direction along the surface that is other than the defined trajectory.

16. A method according to claim 15, wherein the guide further comprising a slider configured to slideably engage the rail, the method further comprising accepting the ultrasound probe to be coupled to the slider.

17. A method according to claim 14, further comprising resisting, but not preventing, some motion even along the defined trajectory, thereby improving ergonomic ability to maintain evenness of movement.

18. A method according to claim 14, further comprising automatically driving the ultrasound probe relative to skin during a scanning operation.

19. A method according to claim 15, further comprising automatically indicating, for a medical ultrasound imaging system, a starting and a finishing of a desired scanning operation.

20. A method of ultrasound image acquisition according to claim 14, further comprising conducting an ultrasound scanning operation, including activating the ultrasound probe, moving the ultrasound probe, receiving signals from the ultrasound probe at a medical ultrasound imaging system to obtain a three dimensional or a panoramic ultrasound image.
